(19) United States
(12) Patent Application Publication
(10) Pub. No.: US 2001/0003043 A1
(43) Pub. Date: Jun. 7, 2001

(54) Method and device for imaging and analysis of biopolymer arrays

(76) Inventors: Andres Metspalu, Tartu (EE); Jevgeni Berik, Tartu (EE); Ants Kurg, Tartu (EE)

Correspondence Address: Eugenia S. Hansen, Sidley & Austin, 717 N. Harwood, Suite 3400, Dallas, TX, 75201-6507, US

(21) Appl. No.: 09/741,960

(22) Filed: Dec. 20, 2000

Related U.S. Application Data
(63) Continuation of application No. PCT/EE00/00001, filed on Apr. 21, 1999.

(30) Foreign Application Priority Data
Apr. 21, 1999 (EE) P 199900072

(51) Int. Cl. C12M 1/34; C12M 3/00; G01J 3/30

(52) U.S. Cl. 435/6; 435/287.2; 356/318

(57) ABSTRACT

The invention disclosed herein is a method and device for parallel detection and analysis of fluorescently labeled biopolymer molecules on a two-dimensional array using lasers for consecutive specific excitation to cause total internal reflection and a charge couple device for emission detection.

### BACKGROUND OF THE INVENTION

Microarrays of short manufactured biopolymers attached onto a solid support in a two-dimensional structure are increasingly used for diagnostic, sequencing, binding, and genome-wide association applications. For imaging and analyzing microarrays, apparatuses using either light detectors or scanning confocal microscopy are used.

One example of the prior art is a fluorescence detector utilizing a charge couple device (CCD) camera called GenoSensor™, manufactured by Vysis, Inc. (Downers Grove, Ill., USA). The GenoSensor™ excites fluorescently-labeled target molecules hybridized to DNA probes bound to a glass support with light traversing the DNA array, as depicted in FIG. 1. The light is generated by a single xenon bulb and passed through one or more filters to select for the spectral band necessary to specifically excite the fluorophore of interest. The light emitted by the fluorophore is filtered and guided through an optical system onto the high-resolution cooled CCD camera. The signals obtained are then processed in a personal computer.

The GenoSensor™, and other similar instruments, have distinct disadvantages for analyzing fluorescently-labeled hybridized microarrays. First, these types of instruments generate significant optical noise because the nucleic acid array is at such a high density that the magnitude of fluorescently-labeled hybridized probes may interfere with the detection of a hybridization event at a single position. Second, using traversing light to excite fluorophores is inefficient because the exciting band must be filtered from the full spectrum. Third, the speed of detection is usually time-consuming where confocal microscopy devices are used because of the scanning mechanism employed. Finally, instruments utilizing white light to excite fluorescently-labeled hybridized microarrays require excitation filters.

The fluorescence detector described herein overcomes the before mentioned disadvantages. The fluorescence detector of the present invention uses total internal reflection to excite a microarray more efficiently than a traversing light beam and obviates the need for a scanning mechanism to excite individual pixels on the microarray. Additionally, the fluorescence detector employs multiple lasers to visualize distinct fluorescently-labeled nucleotides, as is used with the Arrayed Primer Extension (APEX) assay.

APEX is a superior method for analyzing nucleic acid sequence over simple hybridization assays. In hybridization based assays, the target to be analyzed is labeled with a fluorophore and hybridized under stringent conditions to immobilized oligonucleotides. Unfortunately, hybridization - based microarray assays are only as selective as the mismatch intolerance of the hybridization conditions and generally have an unfavorable signal to noise ratio. In contrast, in APEX assays if the hybridization between the immobilized probe and the target is not perfect, the polymerase will neither recognize the structure, nor carry out a reaction. Furthermore, because a fluorescent terminating nucleotide is incorporated onto the primer affixed to the support, a wash of the array after the reaction removes unincorporated fluorescent material to improve the signal to noise ratio in APEX assays. APEX is a better method for analyzing nucleic acid sequence than hybridization assays, but is not as widely used because of the limitations of currently available fluorescent detectors. A fluorescence detector used in conjunction with APEX preferably excites and detects four spectrally distinct fluorophores sequentially. The presently disclosed invention is distinctly configured be used with the APEX assay.

### SUMMARY OF THE INVENTION

The invention described herein is a method and instrument for imaging biopolymer arrays utilizing total internal reflection (FIG. 2) and a fluorescence detecting device enabling a quick and precise analysis of a microarray incorporating multiple distinct spectral bands. The fluorescence detector of the present invention works by directing a beam of light of chosen wavelength into the edge of the support under an angle that will evoke total internal reflection of the beam, making the support into a waveguide (FIG. 2). Despite causing total internal reflection in the waveguide support, a small portion of the internally reflected electromagnetic energy escapes from the surface of the waveguide as an evanescent wave. The intensity of the evanescent wave falls exponentially as the distance the light travels increases, but remains sufficient to excite fluorophores incorporated in the primers bound to the waveguide at a distance of 1/4 of the wavelength If there are four different fluorescently-labeled nucleotides, laser beams of four different wavelengths are used to achieve maximal and specific excitation of each fluorescent label in turn. The light emitted by the fluorophores is gathered through emission filters to discard the background light and focused through an optical system for detection by a charge couple device camera with a high quantum efficiency. As the camera used is cooled, the imaging time is short, taking about 10 seconds for each fluorescence channel. The collected emission spectra are then analyzed on a personal computer.

### BRIEF DESCRIPTION OF DRAWINGS

FIG. 1 is the excitation of fluorophores on the surface of a biopolymer array by traversing light.

FIG. 2 is the excitation by total internal reflection fluorescence.

FIG. 3 is an application wherein the laser beam evokes total internal reflection by being focused through a cylindrical lens so that the diameter of its shape is less than the thickness of the support.

FIG. 4 is an application wherein a prism is used to direct the laser beam into the support. Between the prism and the support there is transparent liquid possessing a refractive index approximately identical to the refractive indices of the prism and the support.

FIG. 5 is the preferred embodiment of the device in this invention, the fluorescence detector.

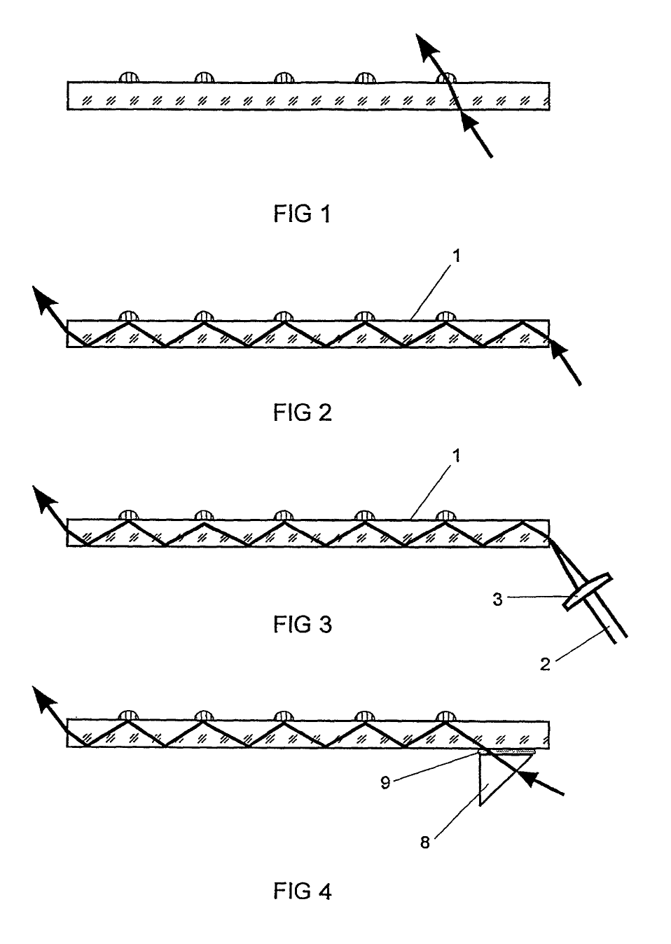

### DETAILED DESCRIPTION OF THE INVENTION

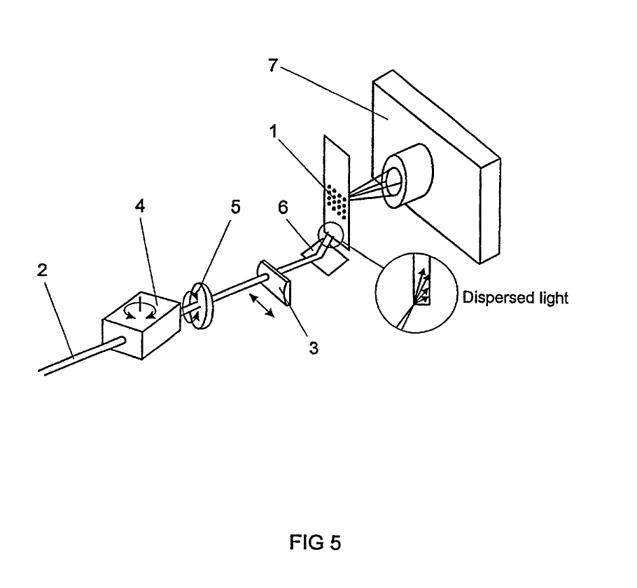

FIGS. 2-5 illustrate the fluorescence detector of the present invention. The invention is a fluorescent detector comprised of a light source capable of specifically and maximally exciting fluorophores located in a biopolymer array on a waveguide support, means for directing the light source into the waveguide support to cause total internal reflection fluorescence on the surface of the waveguide support, and a charge couple device for detecting emission spectra (FIG. 5). The waveguide support (1) is preferably a glass slide, although any transparent material onto which manufactured biopolymers can be affixed and in which total internal reflection can be achieved can be included in the present invention.

The light source (2) is characterized by the ability to excite at least one, and preferably four, spectrally distinct fluorescently-labeled nucleotides. Therefore, the light source could generate one to four spectrally distinct wavelengths of light. Alternatively, the light source could be one to four separate lasers. A diffraction grating may be utilized to decrease background excitation energy.

The means for directing the light source into the waveguide support to cause total internal reflection in the waveguide support is generated in a variety of ways. All components used to focus light from the light source into the waveguide support are designed to make the process of finding the angle under which total internal reflection is generated more efficient and to maximize the most uniform distribution of light in the waveguide support. Therefore, other components may be used interchangeably if they perform the same function of directing the light beam into the waveguide support to generate total internal reflection. One of the components used in the present invention to direct the light beam is a transparent hexahedron (4), which revolves around an axis perpendicular to the light beam. Another component that is used in the present invention to direct the light beam is an optical wedge (5), which revolves around an axis approximating the light beam. A third component is a mirror (6) to reflect the light beam into the waveguide support. Additionally, a prism (8) can be used to direct the light beam into the waveguide support, as depicted in FIG. 4. To minimize the transitional loss of light from the prism to the support, a transparent liquid (9) can be used if its refractive index is approximately equal to the refractive indices of the prism and the waveguide support.

Not only must the light beam enter the waveguide support under a certain angle to generate total internal reflection, but to increase intensity the beam can be focused into a fan shape thinner than the edge of the waveguide support it is entering by a cylindrical lens (3) as in FIG. 3. Presumably, a different component could be substituted for the cylindrical lens if it performs the same function of focusing the light beam into a shape thinner the edge of the waveguide support the light beam is entering.

Emission spectra are detected by a digitally controlled cooled charge-couple device camera (7) and the data stored in a personal computer. Bandwith filters are utilized to decrease the background emission energy from scattered light and extraneous fluorescence. As with other parts of this invention, substituting components which perform the same functions are hereby included in this application.

The fluorescent detector of the present invention is particularly well suited for detecting and analyzing data generated with the APEX method of sequence identification. In APEX, primers of a known sequence are attached at known locations to a solid support which acts as a waveguide. Next, a polynucleotide of interest is hybridized to the array of oligonucleotide primers to generate double stranded oligonucleotides. The double stranded oligonucleotides are incubated with a stringent polymerase and four spectrally unique fluorescently-labeled terminating nucleotides. The primers are then extended by a sequence specific single base polymerization reaction with the addition of a fluorescently-labeled terminating nucleotide to the attached primer. Next, the polynucleotide of interest is melted from the array of oligonucleotide primers to leave only fluorescently-labeled primers on the waveguide support. The microarray is then washed to remove unincorporated fluorescent material to reduce background emission. The waveguide support is then spatially situated between a light source and a charge couple device in the fluorescence detector of the present invention. The light source directed into the waveguide support specifically excites each fluorescently-labeled nucleotide sequentially and emission from the fluorescent nucleotide is detected with a cooled charge couple device. Finally, the emission is analyzed on a personal computer.

Although the invention is described in connection with the practical preferred embodiment, it is understood that the invention is not limited by the prescribed subject matter but intended to include different modifications and equivalents which are comprised in the spirit and scope of the invention.

We claim:

1. A fluorescence detector comprising:
a) a light source for exciting specific fluorophores located on a biopolymer array;
b) means for directing said light source into said waveguide support to cause total internal fluorescence in said waveguide support; and
c) a charge couple device for detecting emission spectra.

2. The fluorescence detector of claim 12, wherein said light source generates a laser beam.

3. The fluorescence detector of claim 12, wherein said light source generates multiple spectrally distinct laser beams.

4. The fluorescence detector of claim 12, wherein said light source is comprised of four spectrally distinct laser beams.

5. The fluorescence detector of claim 12, further comprising a transparent hexahedron, wherein said transparent hexahedron revolves around an axis perpendicular to said light beam for placing said light source into said waveguide support.

6. The fluorescence detector of claim 12, further comprising an optical wedge, wherein said optical wedge revolves around an axis approximating said light beam for placing said light source into said waveguide support.

7. The fluorescence detector of claim 12, further comprising a cylindrical lens for focusing said light beam into a shape smalled than an edge of said waveguide, wherein said light beam is entering said waveguide at said edge.

8. The fluorescence detector of claim 12, further comprising a mirror for directing said light beam into said waveguide support.

9. The fluorescence detector of claim 12, further comprising a diffraction grating for directing said light beam into said waveguide support.

10. The fluorescence detector of claim 12, further comprising an optical prism for directing said light beam into said waveguide support.

11. The fluorescence detector of claim 12, further comprising a transparent liquid placed between said waveguide support and said optical prism, wherein said transparent liquid possesses a refractive index about equal to the refractive indices possessed by said waveguide support and said optical prism.

12. The fluorescence detector of claim 12, wherein said waveguide support has a polished edge in which said light beam enters said waveguide support to illuminate said waveguide support broadly.

13. The fluorescence detector of claim 12, wherein said waveguide support has a frosted edge in which said light beam enters said waveguide support to illuminate said waveguide support broadly.

14. The fluorescence detector of claim 12, further comprising bandpass filters for separating emission spectra.

15. The fluorescence detector of claim 12, further comprising a personal computer to collect and analyze emission spectra.

16. A method for detecting and analyzing a specific nucleic acid sequence comprising:
a) inserting a waveguide support into a fluoresecence detector, said waveguide support being spatially situated between a light source and a charge couple device in said fluorescence detector, wherein said waveguide support possesses an array of affixed oligonucleotides, wherein at least one said oligonucleotide possesses one fluorescent nucleotide;
b) exciting said fluorescent nucleotide by directing said light source to said waveguide support;
c) detecting emission from said fluorescent nucleotide with said charge couple device; and
d) analyzing said emission on a personal computer.

17. A method of analyzing the sequence of a polynucleotide of interest, comprising the steps of:
a) attaching an array of oligonucleotide primers having known sequences to a solid support at known locations, wherein said solid support may act as a waveguide;
b) hybridizing the polynucleotide of interest to the array of oligonucleotide primers to generate double stranded oligonucleotides;
c) subjecting the double stranded oligonucleotides to a sequence specific single base polymerization reaction to extend the annealed primers by the addition of a fluorescently-labelled terminating nucleotide, wherein said primers may be extended by any fluorescently-labelled terminating nucleotide which is complimentary to the polynucleotide of interest;
d) removing the polynucleotide of interest from the array of oligonucleotide primers;
e) inserting said support into a fluoresecence detector, wherein said support is spatially situated between a light source and a charge couple device in said fluorescence detector, wherein said light source is able to specifically excite each fluorescently-labelled nucleotide sequentially;
f) exciting said fluorescent nucleotide by directing said light source into said support;
g) detecting emission from said fluorescent nucleotide with said charge couple device; and
h) analyzing said emission on a personal computer.
